(19) United States
(12) Patent Application Publication
(10) Pub. No.: US 2026/0158465 A1
(43) Pub. Date: Jun. 11, 2026

(54) REACTION DEVICE

(71) Applicant: Panasonic Intellectual Property Management Co., Ltd., Osaka (JP)

(72) Inventors: TAICHI NAKAMURA, Osaka (JP); HIROAKI YOSHIDA, Osaka (JP); YOSHIE TAKAHASHI, Osaka (JP); RIHO MORIYAMA, Osaka (JP)

(21) Appl. No.: 19/535,257

(22) Filed: Feb. 10, 2026

Related U.S. Application Data
(63) Continuation of application No. PCT/JP2024/032602, filed on Sep. 11, 2024.

(30) Foreign Application Priority Data
Oct. 18, 2023 (JP) 2023-179762

(51) Int. Cl. B01J 19/24; B01F 23/40; B01F 23/45; B01F 25/431; B01F 101/00; B01J 14/00; B01J 19/02; C08G 61/10

(52) U.S. Cl. CPC B01J 19/2415; B01F 23/405; B01F 23/45; B01F 25/431; B01J 14/00; B01J 19/02; C08G 61/10; B01F 2101/2204; B01F 2215/0431; B01J 2219/0245; C08G 2261/11; C08G 2261/312; C08G 2261/412

(57) ABSTRACT

A reaction device is a reaction device including a fluid mixer for producing a reaction product by a coupling reaction from raw materials containing at least one type of substrate, a metal catalyst, and an organic solvent, in which the fluid mixer constitutes a liquid contact component that causes a first fluid and a second fluid to flow separately each other, and mixes the first fluid and the second fluid at an end of the liquid contact component, the fluid mixer includes: a first tubular body; and a second tubular body disposed to form a double tube in a portion inside the first tubular body, the first fluid flows between an inner wall of the first tubular body and an outer wall of the second tubular body, and the second fluid flows inside the second tubular body, the first fluid and the second fluid are brought into contact with each other at a tip of the second tubular body of the end of the liquid contact component, the first fluid contains the organic solvent and one of the raw materials dissolved or dispersed in the organic solvent, and the second fluid contains the organic solvent and a remainder of the raw materials dissolved or dispersed in the organic solvent.

### TECHNICAL FIELD

The present disclosure relates in particular to a reaction device for use in coupling reactions using metal catalysts.

### BACKGROUND ART

A synthesis method using a metal catalyst, for example, a palladium catalyst as a heterogeneous catalyst has started to be studied. The heterogeneous catalyst has an advantage that reaction solution and the palladium catalyst can be separated only by filtration after completion of the reaction.

As a coupling reaction using a heterogeneous palladium catalyst, for example, there is a synthesis method for improving the yield of the coupling reaction by using a flow type microreactor filled with a solid palladium catalyst (see, for example, PTL 1).

### CITATION LIST

### Patent Literature

PTL 1: Japanese Patent No. 4778710

### SUMMARY OF THE INVENTION

In the flow type microreactor filled with the solid palladium catalyst, since a velocity distribution occurs at a center and an inner wall of a tube, contact between a reactive substrate and a solid catalyst may vary, and the yield of the coupling reaction may decrease. Therefore, in order to apply the coupling reaction using the heterogeneous catalyst to microflow synthesis, there is a problem that it is necessary to uniformize the contact between the reactive substrate and the solid catalyst while utilizing characteristics of the microflow synthesis of precise reaction control in a minute reaction field.

An object of the present disclosure is to provide a reaction device using a mixer that promotes more excellent contact between the reactive substrate and the solid catalyst while maintaining precise reaction control (specifically, mixing performance and temperature control).

A reaction device according to one aspect of the present disclosure is a reaction device including a fluid mixer for producing a reaction product by a coupling reaction from raw materials containing at least one type of substrate, a metal catalyst, and an organic solvent, in which the fluid mixer constitutes a liquid contact component that causes a first fluid and a second fluid to flow separately each other, and mixes the first fluid and the second fluid at an end of the liquid contact component, the fluid mixer includes: a first tubular body; and a second tubular body disposed to form a double tube in a portion inside the first tubular body, the first fluid flows between an inner wall of the first tubular body and an outer wall of the second tubular body, and the second fluid flows inside the second tubular body, the first fluid and the second fluid are brought into contact with each other at a tip of the second tubular body of the end of the liquid contact component, the first fluid contains the organic solvent and one of the raw materials dissolved or dispersed in the organic solvent, and the second fluid contains the organic solvent and a remainder of the raw materials dissolved or dispersed in the organic solvent.

A production method according to one aspect of the present disclosure is a production method for producing a reaction product by a coupling reaction from raw materials containing at least one type of substrate, a metal catalyst, and an organic solvent, the production method including: causing the raw materials to flow to a liquid contact component including a first tubular body and a second tubular body disposed to form a double tube in a portion inside the first tubular body, the causing the raw materials to flow to the liquid contact component including: causing a first fluid to flow between an inner wall of the first tubular body and an outer wall of the second tubular body, causing a second fluid to flow inside the second tubular body, and causing the first fluid and the second fluid to come into contact with each other at an end of the liquid contact component; and mixing the first fluid and the second fluid in a static mixer including a fluid mixing structure fluidly connected to the liquid contact component, in which the first fluid contains the organic solvent and at least one of the raw materials dissolved or dispersed in the organic solvent, the second fluid contains the organic solvent and a remainder of the raw materials dissolved or dispersed in the organic solvent, and a tip of the second tubular body and the fluid mixing structure are arranged with a gap therebetween.

As described above, according to the reaction device according to the present disclosure, it is possible to obtain a high yield particularly in the coupling reactions using the metal catalysts by promoting more excellent contact between the reactive substrate and the solid catalyst while maintaining precise reaction control (specifically, mixing performance and temperature control).

### BRIEF DESCRIPTION OF THE DRAWINGS

FIG. 1 is a schematic diagram illustrating a configuration of a reaction device according to a first exemplary embodiment.

FIG. 2 is a schematic sectional view illustrating a sectional structure of a fluid mixer according to the first exemplary embodiment.

FIG. 3 is a schematic sectional view illustrating the sectional structure of the fluid mixer according to the first exemplary embodiment.

FIG. 4 is a schematic sectional view illustrating the sectional structure of the fluid mixer of a best mode according to the first exemplary embodiment.

FIG. 5 is an enlarged sectional view taken along a flow direction (an X direction) near a tip of a second tubular body.

FIG. 6 is Table 1 showing yields and determination results of whether liquid can be fed in Example 1, Comparative Example 1, and Comparative Example 2.

### DESCRIPTION OF EMBODIMENT

A reaction device according to a first aspect is a reaction device including a fluid mixer for producing a reaction product by a coupling reaction from raw materials containing at least one type of substrate, a metal catalyst, and an organic solvent, in which the fluid mixer constitutes a liquid contact component that causes a first fluid and a second fluid to flow separately each other, and brings the first fluid and the second fluid into contact with each other at an end of the liquid contact component, the fluid mixer includes: a first tubular body; and a second tubular body disposed to form a double tube in a portion inside the first tubular body, the first fluid flows between an inner wall of the first tubular body and an outer wall of the second tubular body, and the second fluid flows inside the second tubular body, the first fluid and the second fluid are brought into contact with each other at a tip of the second tubular body of the end of the liquid contact component, the first fluid contains the organic solvent and one of the raw materials dissolved or dispersed in the organic solvent, and the second fluid contains the organic solvent and a remainder of the raw materials dissolved or dispersed in the organic solvent.

In the reaction device according to a second aspect, in the first aspect, inner diameter “a” of the second tubular body may be 0.1 mm or more and 10 mm or less.

In the reaction device according to a third aspect, in the first or second aspect, the tip of the second tubular body may have a tapered shape.

The reaction device according to a fourth aspect, in any one of the first to third aspects, may further include a static mixer including a fluid mixing structure that is fluidly connected to the end of the liquid contact component and promotes mixing of the first fluid and the second fluid, and a gap may be provided between the tip of the second tubular body and the fluid mixing structure.

In the reaction device according to a fifth aspect, in the fourth aspect, the second tubular body and the fluid mixing structure may be physically separated from each other by gap length “d”, and the gap length “d” may satisfy the following formula (1).

0.2a≤d≤500a (1)

In the reaction device according to a sixth aspect, in any one of the first to fifth aspects, the at least one type of substrate may have a halogen substituent, and the first tubular body and/or the second tubular body may have stainless steel and a fluororesin coated on a surface of the stainless steel.

A production method according to a seventh aspect is a production method for producing a reaction product by a coupling reaction from raw materials containing at least one type of substrate, a metal catalyst, and an organic solvent, the production method including: causing the raw materials to flow to a liquid contact component including a first tubular body and a second tubular body disposed to form a double tube in a portion inside the first tubular body, the causing the raw materials to flow to the liquid contact component including: causing a first fluid to flow between an inner wall of the first tubular body and an outer wall of the second tubular body, causing a second fluid to flow inside the second tubular body, and causing the first fluid and the second fluid to come into contact with each other at an end of the liquid contact component; and mixing the first fluid and the second fluid in a static mixer including a fluid mixing structure fluidly connected to the liquid contact component, in which the first fluid contains an end organic solvent and at least type of the raw materials dissolved or dispersed in the end organic solvent, the second fluid contains the organic solvent and a remainder of the raw materials dissolved or dispersed in the organic solvent, and the second tubular body includes a tip, and a gap is provided between the tip and the fluid mixing structure.

In the production method according to an eighth aspect, in the seventh aspect, inner diameter “a” of the second tubular body may be 0.1 mm or more and 10 mm or less.

In the production method according to a ninth aspect, in the seventh or eighth aspect, the tip of the second tubular body may have a tapered shape.

In the production method according to a tenth aspect, in any one of the seventh to ninth aspects, the second tubular body and the fluid mixing structure may be physically separated from each other by gap length “d”, and the gap length “d” may satisfy the following formula (1).

0.2a≤d≤500a (1)

In the production method according to an eleventh aspect, in any one of the seventh to tenth aspects, the at least one type of substrate may have a halogen substituent, and the first tubular body and/or the second tubular body may have stainless steel and a fluororesin coated on a surface of the stainless steel.

Hereinafter, a reaction device and a production method of a reaction product using the reaction device according to an exemplary embodiment will be described with reference to the accompanying drawings. However, unless otherwise specified, constituent elements, types, combinations, shapes, relative positions, and the like described in the exemplary embodiment are not intended to limit the scope of the present disclosure only thereto, and are merely illustrative examples.

### First Exemplary Embodiment

### <Reaction Device 100>

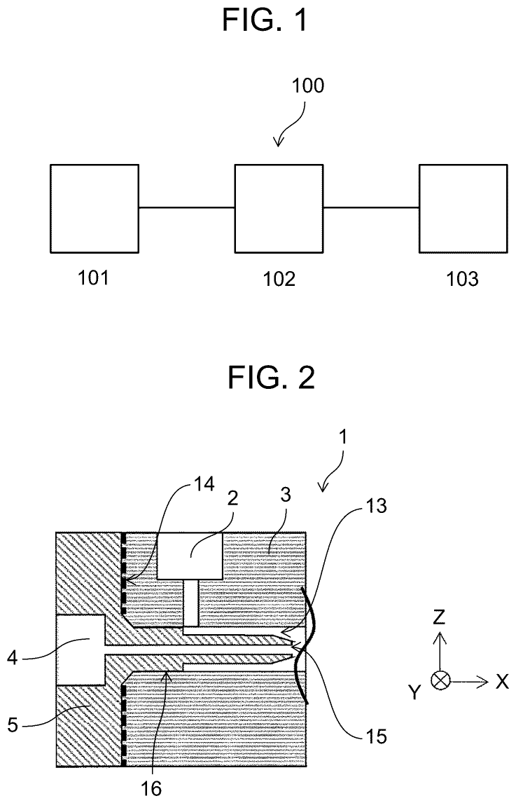

FIG. 1 is a schematic diagram illustrating a configuration of reaction device 100 according to a first exemplary embodiment.

Reaction device 100 according to the first exemplary embodiment includes raw material supply unit 101, reaction unit 102, and recovery unit 103. Raw material supply unit 101 transfers the raw materials containing at least one type of substrate, a metal catalyst, and an organic solvent using at least two or more liquid feeding units. Reaction unit 102 uses fluid mixer 1 to be described later, to mix the raw materials transferred from raw material supply unit 101, and appropriately maintain the raw materials in a constant temperature state to react. Recovery unit 103 recovers a compound produced by a desired reaction in reaction unit 102.

### <Fluid Mixer 1>

FIG. 2 is a schematic sectional view illustrating a sectional structure of fluid mixer 1 according to the first exemplary embodiment. Note that, for convenience, a direction of flow of the fluid is indicated as an X direction, a direction from the front to the back of the paper surface among directions perpendicular to the X direction is indicated as a Y direction, and a direction from the bottom to the top of the paper surface is indicated as a Z direction.

Fluid mixer 1 according to the first exemplary embodiment includes first tubular body 3 and second tubular body 5 disposed to form the double tube in a portion inside first tubular body 3. The first fluid can flow between the inner wall of first tubular body 3 and the outer wall of second tubular body 5. The second fluid can flow inside second tubular body 5. Fluid mixer 1 includes first pipe connection portion 2 for transporting the first fluid to first tubular body 3. Fluid mixer 1 further includes second pipe connection portion 4 for transporting the second fluid to second tubular body 5. At the tip of second tubular body 5, the first fluid and the second fluid come into contact with each other to start mixing of two fluids, and mixing of the fluids and the resulting reaction further proceed by an extended pipe (not illustrated) located downstream.

Next, components of this fluid mixer 1 will be described.

As described above, fluid mixer 1 includes first tubular body 3 and second tubular body 5 disposed to form the double tube in a portion inside first tubular body 3. Fluid mixer 1 constitutes liquid contact component 6 that separates the raw materials into the first fluid and the second fluid, causes the first fluid and the second fluid to flow through liquid contact component 6, and mixes the first fluid and the second fluid at an end of liquid contact component 6. Liquid contact component 6 includes first pipe connection portion 2 for transporting the first fluid to first tubular body 3. Liquid contact mixer 6 further includes second pipe connection portion 4 for transporting the second fluid to second tubular body 5.

### <First Pipe Connection Portion 2>

First pipe connection portion 2 connects a pipe for transporting the first fluid and first tubular body 3. As first pipe connection portion 2, a member such as a tapered screw, a parallel screw, or a hose joint can be used.

### <First Tubular Body 3>

First tubular body 3 has cavity 13 capable of transporting the first fluid inside first tubular body 3. First tubular body 3 receives a portion of second tubular body 5 inside cavity 13. As first tubular body 3, a stainless steel such as SUS304, SUS316, or SUS316L, a metal material such as HASTELLOY, a processed resin material such as PP, PFA, PTFE, PEEK, or PPS, tubes made of these materials, or the like can be used, however, when reaction unit 102 is maintained in the constant temperature state, the stainless steel is desirable in order to ensure heat transfer from a heat source. However, when at least one type of substrate of the raw materials has a halogen substituent, it is desirable to coat the surface of the stainless steel with the fluororesin from the viewpoint of corrosion resistance. A section perpendicular to an axis of cavity 13 may be any of an elliptical shape, a circular shape, a polygonal shape, and the like, but is, for example, the circular shape.

### <Second Pipe Connection Portion 4>

Second pipe connection portion 4 connects a pipe for transporting the second fluid and second tubular body 5. As second pipe connection portion 4, the member such as a tapered screw, a parallel screw, or a hose joint can be used.

### <Second Tubular Body 5>

Second tubular body 5 has cavity 15 capable of transporting the second fluid inside second tubular body 5. Furthermore, in cavity 13 inside first tubular body 3, a portion outside second tubular body 5 only needs to be in contact with the first fluid. As second tubular body 5, the stainless steel such as SUS304, SUS316, or SUS316L, the metal material such as HASTELLOY, the processed resin material such as PP, PFA, PTFE, PEEK, or PPS, the tubes made of these materials, or the like can be used, however, when reaction unit 102 is maintained in the constant temperature state, the stainless steel is desirable in order to ensure heat transfer from the heat source. However, when at least one type of substrate of the raw materials has a halogen substituent, it is desirable to coat the surface of the stainless steel with the fluororesin from the viewpoint of corrosion resistance. A section perpendicular to an axis of cavity 15 may be any of the elliptical shape, the circular shape, the polygonal shape, and the like, but is, for example, the circular shape. When a sectional shape with respect to the axis of cavity 15 is circular, a contact interface between the first fluid and the second fluid can be formed more accurately in axial symmetry.

Inner diameter a of cavity 15 of second tubular body 5 is preferably 0.1 mm or more and 10 mm or less. Inner diameter a of 0.1 mm or more is desirable from the viewpoint of reducing pressure loss in a flow path to promote liquid feeding and producing a material. Inner diameter a of 0.1 mm or more is also desirable from the viewpoint of processing accuracy at the time of preparing second tubular body 5. Inner diameter a of 10 mm or less is desirable because the second fluid flowing near a center of cavity 15 of second tubular body 5 is mixed with the first fluid in a short time to improve mixing performance of the fluid mixer.

### <Arrangement of First Tubular Body 3 and Second Tubular Body 5>

As illustrated in FIG. 1, first tubular body 3 includes a portion of second tubular body 5 in cavity 13 inside first tubular body 3. That is, the first tubular body 3 and the second tubular body 5 are arranged to be a double tube structure such that an axial direction (the X direction) of cavity 13 of first tubular body 3 coincides with an axial direction (the X direction) of cavity 15 of second tubular body 5.

Conventionally, in such a double tube structure, there have been many cases where two tubular bodies are connected by threading inner surface 16 in contact in parallel in the axial direction. However, in this case, concentricity of a double tube portion is not sufficient.

In fluid mixer 1 according to the exemplary embodiment, first tubular body 3 and second tubular body 5 are connected not by inner surface 16 parallel to the axial direction (X direction) but by a surface intersecting the axial direction (X direction), for example, vertical end surface 14 in FIG. 1. Thus, alignment between the first tubular body 3 and the second tubular body 5 on a plane (Y-Z plane) perpendicular to the axial direction (X direction) can be performed with higher accuracy, and sufficient concentricity can be secured. A connection surface between the first tubular body 3 and the second tubular body 5 is not limited to the plane perpendicular to the axial direction (X direction), and may be any surface intersecting the axial direction (X direction). For example, it is sufficient if the connection surface intersects the axial direction (X direction) at an angle of 30 degrees or more. Furthermore, it may intersect the axial direction at an angle of 45 degrees or more.

Thus, the concentricity between the axis of cavity 13 of first tubular body 3 and the axis of cavity 15 of second tubular body 5 can be made sufficient.

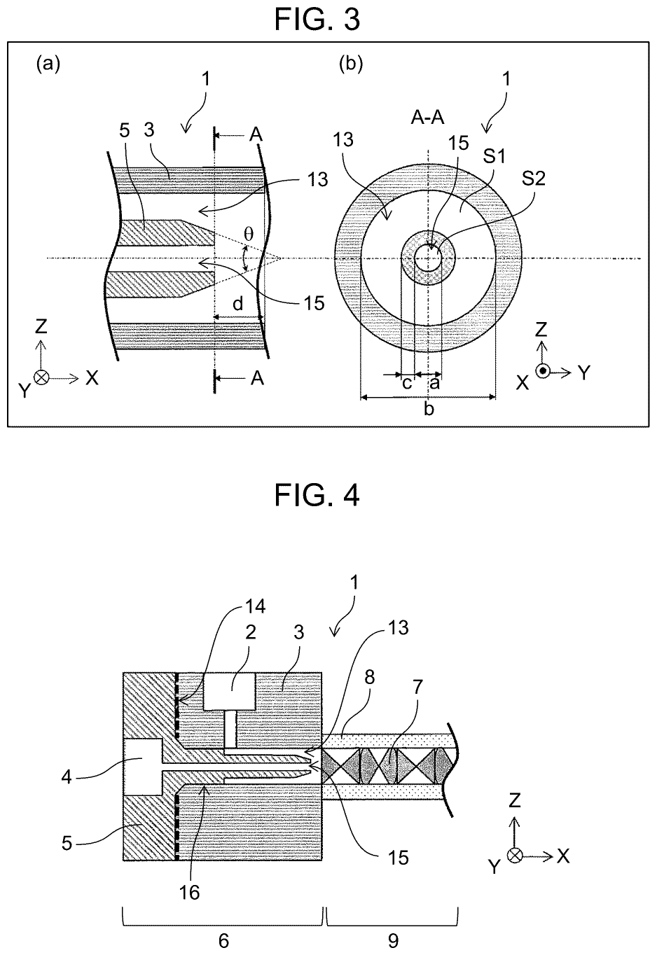

Next, detailed dimensions of the fluid mixer will be described with reference to FIG. 3.

Part (a) of FIG. 3 is an enlarged sectional view taken along the flow direction (X direction) near the tip of second tubular body 5, and part (b) of FIG. 3 is an enlarged sectional view perpendicular to the flow direction.

### <Relationship Between Tip of Second Tubular Body 5 and Extended Pipe (not Illustrated) Located Downstream>

The tip of second tubular body 5 and the extended pipe (not illustrated) located downstream are physically separated from each other by gap length d. Gap length d satisfies the following formula (1), which is a relational expression with inner diameter a of second tubular body 5.

$$0.2 a \leq d \leq 500 a \qquad ( 1 )$$

The extended pipe (not illustrated) located downstream can accumulate a product. Gap length d of 0.2a or more prevents the accumulated product from reaching second tubular body 5, and prevents second tubular body 5 from being blocked. On the other hand, gap length d of 500a or less shortens a time from contact between the first fluid and the second fluid to complete mixing, and improves the mixing performance of fluid mixer 1.

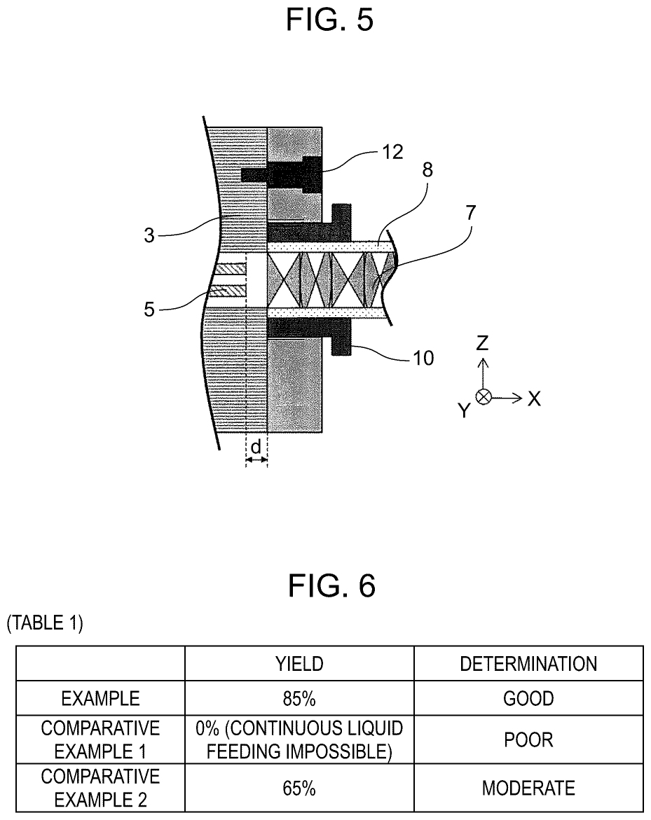

The extended pipe located downstream may be, for example, fitting portion 10 and third tubular body 8 that is a tube, which do not include the fluid mixing structure connected downstream of the fluid mixer of FIG. 5 described later.

In the fluid mixer according to the first exemplary embodiment, the tip of the second tubular body is provided so as not to be in physical contact with third tubular body 8. This makes it possible to achieve both prevention of the product from adhering to the flow path and removal of the product, and the mixing performance.

### <Relationship Between First Tubular Body 3 and Tip of Second Tubular Body 5>

The tip of second tubular body 5 is preferably tapered. Since the tip of second tubular body 5 is tapered, a shearing force due to flow of a cylinder center direction component (the X direction) of the second fluid is generated at a contact portion between the interface between the first fluid and the second fluid and second tubular body 5, and the product adhering to the contact portion between the interface between the first fluid and the second fluid and second tubular body 5 is easily removed. Even when the concentricity of first tubular body 3 and second tubular body 5 does not match with high accuracy, the first fluid and the second fluid can be stably brought into contact with each other without the tip of second tubular body 5 coming into contact with first tubular body 3, and adhesion of the product to the flow path can be further reduced. Taper angle θ of the tapered shape of the tip of second tubular body 5 is preferably 10° or more and 150° or less, and more preferably 15° or more and 90° or less. Taper angle θ of 10° or more enhances an effect of removing attached substances of the shearing force caused by the flow of the cylinder center direction component (X direction) of the second fluid, and thus a desired effect is easily exhibited. On the other hand, since taper angle θ of 150° or less prevents a tapered portion from being a stagnation point, the product generated at the interface between the first fluid and the second fluid is less likely to adhere to the tapered portion, and the desired effect is easily exhibited.

In addition, it is preferable that sectional area S2 and area S1 illustrated in part (b) of FIG. 3 satisfy the following formula (2). Sectional area S2 is a sectional area of an inner peripheral portion of second tubular body 5 at an end surface of the tip of second tubular body 5. Area S1 is an area obtained by subtracting a sectional area of an outer peripheral portion of second tubular body 5 from a sectional area of an inner peripheral portion of first tubular body 3.

$$S 2 / S 1 \leq 3.09 \qquad ( 2 )$$

The contact between the first fluid and the second fluid produces the product at the interface between the first fluid and the second fluid. Since the first fluid and the second fluid come into contact with each other at a position away from the inner wall of first tubular body 3, the product can be prevented from adhering to the inner wall of first tubular body 3. S2/S1 of 3.09 or less is desirable because the interface between the first fluid and the second fluid is separated from the inner wall of first tubular body 3, and it reduces the possibility that the product adheres to the inner wall of first tubular body 3 due to diffusion or precipitation. Inner diameter a of second tubular body 5 is desirably 0.1 mm or more from the viewpoint of the processing accuracy of the member, and inner diameter b of first tubular body 3 is desirably 20 mm or less from the viewpoint of the mixing performance. Therefore, a value of S2/S1 is preferably 2.5×10⁻⁵ or more, 2.5×10⁻⁵ being a numerical value corresponding to a case where inner diameter a of second tubular body 5 is 0.1 mm, inner diameter b of first tubular body 3 is 20 mm, and tip thickness c of second tubular body 5 is 0.2 mm.

Next, as a configuration of fluid mixer 1, a best mode used when the mixing performance is not secured in a simple extended pipe located downstream of the tip of second tubular body 5 and the desired reaction cannot be performed will be described with reference to FIG. 4.

Fluid mixer 1 of the best mode according to the first exemplary embodiment includes liquid contact component 6 that separates the raw materials into the first fluid and the second fluid, causes the first fluid and the second fluid to flow through liquid contact component 6, and mixes the first fluid and the second fluid at the end of liquid contact component 6. Liquid contact component 6 includes first tubular body 3 and second tubular body 5 disposed to form the double tube in a portion inside first tubular body 3. The first fluid can flow between the inner wall of first tubular body 3 and the outer wall of second tubular body 5. The second fluid can flow inside second tubular body 5. The first fluid and the second fluid come into contact with each other at the tip of the second tubular body at the end of liquid contact component 6. Liquid contact component 6 includes first pipe connection portion 2 for transporting the first fluid to first tubular body 3. Liquid contact component 6 further includes second pipe connection portion 4 for transporting the second fluid to second tubular body 5.

Fluid mixer 1 may include static mixer 9 with fluid mixing structure 7 fluidly connected to the end of liquid contact component 6 to facilitate mixing of the first fluid and the second fluid. Static mixer 9 further includes third tubular body 8 that houses fluid mixing structure 7 therein. At the tip of second tubular body 5, the first fluid and the second fluid come into contact with each other to start mixing of the two fluids, and the mixing of the fluids further proceeds by fluid mixing structure 7 located downstream.

### <Static Mixer 9>

Static mixer 9 includes fluid mixing structure 7 fluidly connected to liquid contact component 6 to facilitate the mixing of the first fluid and the second fluid. Static mixer 9 further includes third tubular body 8 that houses fluid mixing structure 7 therein.

### <Fluid Mixing Structure 7>

Fluid mixing structure 7 may be any structure as long as the mixing of the fluids is promoted by stirring or diffusion, and it is possible to use a structure using a stirring blade-like structure or a structure in which fluid division and joining are repeated a plurality of times.

### <Third Tubular Body 8>

Third tubular body 8 is only required to be able to transport a mixture of the first fluid and the second fluid, have fluid mixing structure 7 therein, and be able to be connected to second tubular body 5. As third tubular body 8, the stainless steel such as SUS304, SUS316, or SUS316L, the metal material such as HASTELLOY, the processed resin material such as PP, PFA, PTFE, PEEK, or PPS, the tubes made of these materials, or the like provided with a connection portion can be used.

Next, detailed dimensions of the fluid mixer of the best mode will be described with reference to FIG. 5.

FIG. 5 is an enlarged sectional view taken along the flow direction (X direction) near the tip of second tubular body 5.

### <Relationship Between Tip of Second Tubular Body 5 and Fluid Mixing Structure 7>

The tip of second tubular body 5 and fluid mixing structure 7 are physically separated from each other by gap d. Gap length d satisfies the following formula (1), which is a relational expression with inner diameter a of second tubular body 5.

$$0.2 a \leq d \leq 500 a \qquad ( 1 )$$

Fluid mixing structure 7 can accumulate the product. Gap length d of 0.2a or more prevents the accumulated product from reaching second tubular body 5, and prevents second tubular body 5 from being blocked. On the other hand, gap length d of 500a or less shortens a time from contact between the first fluid and the second fluid to complete mixing, and improves the mixing performance of fluid mixer 1.

In fluid mixer 1 of the best mode according to the exemplary embodiment, the tip of the second tubular body is provided so as not to be in physical contact with the fluid mixing structure. This makes it possible to achieve both prevention of the product from adhering to the flow path and removal of the product, and the mixing performance.

Hereinafter, an effect of applying the coupling reaction to promote the contact between the reactive substrate and the solid catalyst by the reaction device using the fluid mixer according to the present exemplary embodiment will be verified and described in detail.

### Example 1

### <Method for Producing Fluid Mixer 1>

As a pipe for transporting the first fluid, a PFA tube having an inner diameter of 1 mm and an outer diameter of 1.59 mm to which fitting 10 of ¼-28UNF was attached was used.

First pipe connection portion 2 was prepared by processing first tubular body 3 with a ¼-28UNF thread standard, and a pipe for transporting the first fluid was connected thereto.

First tubular body 3 was prepared by processing a column made of SUS316 into a cylindrical shape with an inner diameter of 2.18 mm.

As a pipe for transporting the second fluid, a PFA tube having an inner diameter of 1 mm and an outer diameter of 1.59 mm to which fitting 10 of ¼-28UNF was attached was used.

Second pipe connection portion 4 was prepared by processing second tubular body 5 with the ¼-28UNF thread standard, and a pipe for transporting the second fluid was connected thereto.

Second tubular body 5 is formed by processing a column made of SUS316, and has an inner diameter of 0.5 mm, a part of the tip has an outer diameter of 2.18 mm so as to be inserted into a part of first tubular body 3, and further an outer diameter of second tubular body 5 at the tip is 0.9 mm, and second tubular body 5 has a tip thickness of 0.2 mm. Second tubular body 5 was prepared such that a distance between the tip of first tubular body 3 and one of end surfaces of second tubular body 5 was 2.18 mm when second tubular body 5 was assembled with first tubular body 3.

Liquid contact component 6 was prepared by assembling first tubular body 3 and second tubular body 5 so that first tubular body 3 and second tubular body 5 were in contact with each other at an end surface perpendicular to a longitudinal direction of a cylinder, and fastening the end surface with a screw.

As a pipe downstream of liquid contact component 6, a PFA tube having an inner diameter of 2.18 mm and an outer diameter of 3.18 mm to which fitting 10 of ¼-28UNF was attached was used.

### <Reductive Homo-Coupling Polymerization>

1. Substrate dichlorobenzene (3.05 mmol)
2. Metal catalyst Ni(COD)₂ (7.27 mmol)
3. Organic solvent DMAc (7.3 ml)
4. Initiator triphenylphosphine (7.25 mmol)

Reductive homo-coupling polymerization shown in Chemical formula 1 was performed with the above raw materials.

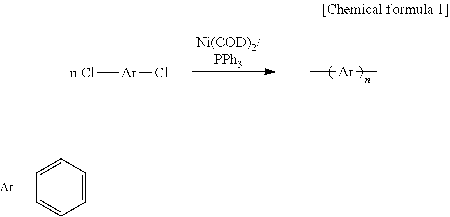

In Example 1, solution A in which a substrate and an initiator were dissolved in an organic solvent was fed to a first pipe of the fluid mixer at a flow rate of 1 mL/min using plunger pump (UI-22) manufactured by FLOM Corporation.

Solution B in which a metal catalyst pulverized so as to have an average particle size of 1 μm or less was dispersed in an organic solvent was fed to a second pipe of the fluid mixer at a flow rate of 1 mL/min using a transfer pump (3HMC025F) manufactured by HEISHIN Ltd.

Dimensions of the liquid contact component of the fluid mixer were set to inner diameter a of 0.5 mm, inner diameter b of 2.18 mm, tip thickness c of 0.05 mm, a taper angle of 30°, and gap length d of 2.18, and a static mixer (K2-24-M) manufactured by NORITAKE CO., LTD. was used as the static mixer.

Note that the fluid mixer was maintained in the constant temperature state so as to have a constant temperature.

The reaction was carried out at a residence time of 20 minutes to obtain a compound.

### Comparative Example 1

Solution A and solution B similar to those in Example 1 were fed using a liquid feeding pump, and the constant temperature state was maintained to be the constant temperature using a three-way joint having an inner diameter of 0.3 mm manufactured by FLOM Corporation instead of the fluid mixer.

The reaction was carried out at a residence time of 20 minutes to obtain a compound.

### Comparative Example 2

Solution C in which a substrate, an initiator, and an organic solvent were dissolved was fed to a syringe filled with a metal catalyst pulverized so as to have an average particle diameter of 1 μm or less at 2 mL/min using plunger pump (UI-22) manufactured by FLOM Corporation.

Note that the syringe was maintained in the constant temperature state so as to have a constant temperature.

The reaction was carried out at a residence time of 20 minutes to obtain a compound.

### (Results)

Table 1 of FIG. 6 shows yields and determination results of whether liquid can be fed in Example 1, Comparative Example 1, and Comparative Example 2.

Comparing Example 1 with Comparative Example 1, in Comparative Example 1, a liquid feeding error of the pump due to an increase in pressure loss occurred during the reaction in the three-way joint, and continuous liquid feeding became impossible. In contrast, in Example 1, by using the fluid mixer according to the present exemplary embodiment, the continuous liquid feeding could be performed and a compound was obtained in a relatively high yield of 85%. In addition, an effect of promoting excellent contact between the reactive substrate and the solid catalyst while maintaining precise reaction control (specifically, mixing performance and temperature control) by blockage suppression was obtained.

Next, comparing Example 1 with Comparative Example 2, in Comparative Example 2, in the syringe filled with the metal catalyst, since a velocity distribution occurred at the center and the inner wall of the tube, the contact between the reactive substrate and the solid catalyst varied, and the yield was only moderate. In contrast, in Example 1, by using the fluid mixer according to the present exemplary embodiment, the compound was obtained in a relatively high yield of 85%. In addition, an effect of promoting excellent contact between the reactive substrate and the solid catalyst while maintaining precise reaction control (specifically, mixing performance and temperature control) by blockage suppression was obtained.

### INDUSTRIAL APPLICABILITY

The reaction device according to the present disclosure has the effect of promoting more excellent contact between the reactive substrate and the solid catalyst while maintaining precise reaction control (specifically, mixing performance and temperature control). Therefore, the present disclosure can be expected to be applied not only to organic synthesis applications such as coupling reactions, but also to mixers for use in synthesizing fine particles and proteins and for use in recycling raw materials in which elements dissolved in a solvent are precipitated and recovered as a solid content.

### REFERENCE MARKS IN THE DRAWINGS

1 fluid mixer
2 first pipe connection portion
3 first tubular body
4 second pipe connection portion
5 second tubular body
6 liquid contact component
7 fluid mixing structure
8 third tubular body
9 static mixer
10 fitting portion
12 screw
13 cavity
14 end surface
15 cavity
16 inner surface
100 reaction device
101 raw material supply unit
102 reaction unit
103 recovery unit

1. A reaction device comprising:
a fluid mixer for producing a reaction product by a coupling reaction from raw materials containing at least one type of substrate, a metal catalyst, and an organic solvent, wherein
the fluid mixer constitutes a liquid contact component that causes a first fluid and a second fluid to flow separately each other, and brings the first fluid and the second fluid into contact with each other at an end of the liquid contact component,
the fluid mixer includes:
a first tubular body; and
a second tubular body disposed to form a double tube in a portion inside the first tubular body,
the first fluid flows between an inner wall of the first tubular body and an outer wall of the second tubular body, and the second fluid flows inside the second tubular body,
the first fluid and the second fluid are brought into contact with each other at a tip of the second tubular body of the end of the liquid contact component,
the first fluid contains the organic solvent and one of the raw materials dissolved or dispersed in the organic solvent, and
the second fluid contains the organic solvent and a remainder of the raw materials dissolved or dispersed in the organic solvent.

2. The reaction device according to claim 1, wherein an inner diameter “a” of the second tubular body is 0.1 mm or more and 10 mm or less.

3. The reaction device according to claim 1, wherein the tip of the second tubular body has a tapered shape.

4. The reaction device according to claim 1, further comprising a static mixer including a fluid mixing structure that is fluidly connected to the end of the liquid contact component and promotes mixing of the first fluid and the second fluid, wherein
a gap is provided between the tip of the second tubular body and the fluid mixing structure.

5. The reaction device according to claim 4, wherein
the second tubular body and the fluid mixing structure are physically separated from each other by a gap length “d”, and
the gap length “d” satisfies the following formula (1).
$$0.2 a \leq d \leq 500 a \qquad ( 1 )$$

6. The reaction device according to claim 1, wherein
the at least one type of substrate has a halogen substituent, and
the first tubular body and/or the second tubular body has stainless steel and a fluororesin coated on a surface of the stainless steel.

7. A production method for producing a reaction product by a coupling reaction from raw materials containing at least one type of substrate, a metal catalyst, and an organic solvent, the production method comprising:
causing the raw materials to flow to a liquid contact component including a first tubular body and a second tubular body disposed to form a double tube in a portion inside the first tubular body,
the causing the raw materials to flow to the liquid contact component including:
causing a first fluid to flow between an inner wall of the first tubular body and an outer wall of the second tubular body,
causing a second fluid to flow inside the second tubular body, and
causing the first fluid and the second fluid to come into contact with each other at an end of the liquid contact component; and
mixing the first fluid and the second fluid in a static mixer including a fluid mixing structure fluidly connected to the liquid contact component, wherein
the first fluid contains the organic solvent and at least one of the raw materials dissolved or dispersed in the organic solvent,
the second fluid contains the organic solvent and a remainder of the raw materials dissolved or dispersed in the organic solvent, and
the second tubular body includes a tip, and a gap (d) is provided between the tip and the fluid mixing structure.

8. The production method according to claim 7, wherein an inner diameter “a” of the second tubular body is 0.1 mm or more and 10 mm or less.

9. The production method according to claim 7, wherein the tip of the second tubular body has a tapered shape.

10. The production method according to claim 7, wherein
the second tubular body and the fluid mixing structure are physically separated from each other by a gap length “d”, and
the gap length “d” satisfies the following formula (1).
$$0.2 a \leq d \leq 500 a \qquad ( 1 )$$

11. The production method according to claim 7, wherein
the at least one type of substrate has a halogen substituent, and
the first tubular body and/or the second tubular body has stainless steel and a fluororesin coated on a surface of the stainless steel.
